(19) United States
(12) Patent Application Publication
(10) Pub. No.: US 2012/0143027 A1
(43) Pub. Date: Jun. 7, 2012

(54) FIELD EFFECT TRANSISTORS FOR DETECTION OF NOSOCOMIAL INFECTION

(75) Inventors: Erica M. Phillips, Woodstock, GA (US); Richard Hantke, Chicago, IL (US); Daniel Baird, Woodstock, GA (US); Mike Rainone, Palestine, TX (US); Thomas Edward Plowman, Cary, NC (US); Talbot Presley, Palestine, TX (US)

(73) Assignee: KIMBERLY-CLARK WORLDWIDE, INC., Neenah, WI (US)

(21) Appl. No.: 11/959,823

(22) Filed: Dec. 19, 2007

(51) Int. Cl. A61B 5/1473

(52) U.S. Cl. 600/345

(57) ABSTRACT

Disclosed herein are methods and devices for detection of hospital acquired infections. Disclosed methods may be utilized for continuous in vivo monitoring of a potential infection site and may be utilized to alert patients and/or health care providers to changes in the local environment due to the presence of a pathogen at an early stage of infection. Disclosed methods utilize ion sensitive field effect transistors (ISFETs) to detect changes in ionic concentration at the site due to the presence of a pathogen, for instance at a surgical site. When a pathogen is present, the local ionic concentration, and hence the electrical characteristics of an ISFET may change, causing a detectable signal from the ISFET. An ISFET may be associated with a biological material such as an enzyme or a specific binding partner for an expression product of a pathogen to improve detection. Upon interaction of the expression product with the enzyme or the probe, the electrical characteristics of the ISFET may change, detection of which may then provide information as to the existence a pathogen at the site.

### BACKGROUND

Nosocomial or hospital acquired infections (HAI) have been estimated by the World Health Organization (WHO) to kill between 1.5 and 3 million people every year worldwide. Though commonly referred to as hospital acquired infections, nosocomial infections result from treatment in any healthcare service unit, and are generally defined as infections that are secondary to the patient's original condition. In the United States, HAIs are estimated to occur in 5 percent of all acute care hospitalizations, resulting in more than $4.5 billion in excess health care costs. According to a survey of U.S. hospitals by the Centers for Disease Control and Prevention (CDC), HAIs accounted for about 1.7 million infections and about 99,000 associated deaths in 2002. The CDC reported that “[t]he number of HAIs exceeded the number of cases of any currently notifiable disease, and deaths associated with HAIs in hospitals exceeded the number attributable to several of the top ten leading causes of death in U.S. vital statistics” (Centers for Disease Control and Prevention, “Estimates of Healthcare Associated Diseases,” May 30, 2007).

HAIs, including surgical site infections (SSIs), catheter related blood stream infections (CRBSIs), urinary tract infections (UTIs), ventilator associated pneumonia (VAP), and others, may be caused by bacteria, viruses, fungi, or parasites. For instance, bacterial organisms, such as Escherichia coli, Staphylococcus aureus, and Pseudomonas aeruginosa are common causes as are yeasts such as Candida albicans and Candida glabrata, fungi such as those of the genus Aspergillus and those of the genus Saccharomyces, and viruses such as parainfluenza and norovirus.

Ongoing efforts are being made to prevent HAI through, for instance, improved hand washing and gloving materials and techniques, but such efforts have met with limited success. In an effort to better understand and curb HAIs, government regulations have increased pressure on hospitals and care-givers to monitor and report these types of infections. However, these measures are further complicated due to the prevalence of outpatient services, a result of which being that many HAIs do not become evident until after the patient has returned home. As such, infection may proceed undiagnosed for some time, complicating treatment and recovery.

Accordingly, there is considerable interest in methods for detecting, measuring and monitoring for early signs of HAI. Chemical sensors belonging to a class of devices known as Chemically Sensitive Field-Effect Transistors (CHEMFET's) are of particular interest for such biomedical applications. CHEMFETs measure chemical properties of the samples to which the device is exposed. In a CHEMFET, changes at the surface of the gate dielectric are detected via modulations of the electric field in the channel of the field-effect transistor. Such chemical changes may be induced by the presence of ions in aqueous solutions. Other changes may be induced by the interaction of an organic compound with a biological-sensing element in contact with the gate of the field-effect transistor. In this way, the concentration of ions or organic biomolecules (e.g., glucose, cholesterol, and so forth) in aqueous solutions may be measured.

Among CHEMFET devices, ion-sensitive field-effect transistors are well known. The concept of ion-sensitive field-effect transistor (ISFET) was introduced by P. Bergveld in 1970 [P. Bergveld, IEEE Trans. Biomed. Eng., BME-17, 1970, pp. 70]. It was demonstrated that when the metal gate of an ordinary MOSFET was omitted and the dielectric layer was exposed to an electrolyte, the characteristics of the transistor were affected by the ionic activity of the electrolyte.

Electrochemical-based detection techniques, and in particular techniques utilizing ISFET-based devices, for early diagnosis of HAI would be of benefit in the art. For instance, a correlation between local pH and the presence of pathogens in vivo has been previously examined (see, e.g., Ye, Plastic & Reconstructive Surgery, 19:3, 213, 1957; Kaufman, et al., Burns, 9, 84, 1978). Methods and devices for early diagnosis of infection through detection of this phenomena would provide great advantage to the art.

### SUMMARY

In accordance with one embodiment, a method for detecting a pathogen that is source of a hospital acquired infection is disclosed. A method may include locating an ion sensitive field effect transistor in an in vivo environment, transmitting an output signal from the ion sensitive field effect transistor to a detector, and detecting a change in the output signal due to the presence of the pathogen in the local in vivo environment.

According to another embodiment, a device for detecting a pathogen that is a source of a hospital acquired infection is disclosed. A device may include a power source and a biocompatible ion sensitive field effect transistor for inserting into an in vivo environment. The ISFET is in electrical communication with the power source. A device may also include a detector in electrical communication with the ISFET. The detector may be utilized to detect a change in the output signal of the biocompatible ISFET due to the presence of a pathogen in the local in vivo environment. In addition, a device may include a signal processor in electrical communication with the detector and a signaling device in electrical communication with the signal processor for emitting a signal upon detection of a change in the output of the ISFET in the local in vivo environment.

Other features and aspects of the present disclosure are discussed in greater detail below.

### BRIEF DESCRIPTION OF THE DRAWINGS

A full and enabling disclosure of the subject matter, including the best mode thereof, directed to one of ordinary skill in the art, is set forth more particularly in the remainder of the specification, which makes reference to the appended figures in which:

FIG. 1 is a schematic representation of an ion-sensitive field effect transistor (ISFET) that may be utilized according to one embodiment of the disclosed subject matter;

FIG. 2 is a schematic representation of an ISFET as may be utilized in one embodiment of the present invention including one or more enzymes immobilized in a layer applied at the gate of the ISFET;

FIG. 3 is a schematic representation of an ISFET as may be utilized in another embodiment of the present invention including a static enzyme sensor;

FIG. 4 is a schematic representation of an ISFET as may be utilized in one embodiment of the present invention including a probe immobilized at the gate;

FIG. 5 is a schematic representation of an ISFET as may be utilized in one embodiment of the present invention including a probe immobilized on an insulator surface of the ISFET; and

FIG. 6 illustrates one embodiment of a system as disclosed herein including a plurality of ISFETs in series.

Repeat use of reference characters in the present specification and drawings is intended to represent same or analogous features or elements.

### DETAILED DESCRIPTION OF REPRESENTATIVE EMBODIMENTS

Reference now will be made in detail to various embodiments of the disclosed subject matter, one or more examples of which are set forth below. Each example is provided by way of explanation, not limitation. In fact, it will be apparent to those skilled in the art that various modifications and variations may be made in the present disclosure without departing from the scope or spirit of the subject matter. For instance, features illustrated or described as part of one embodiment, may be used on another embodiment to yield a still further embodiment. Thus, it is intended that the present disclosure covers such modifications and variations as come within the scope of the appended claims and their equivalents.

The present disclosure is generally directed to methods for detection of HAI, i.e., nosocomial infection. In one embodiment, disclosed methods may be utilized for continuous in vivo monitoring of a potential infection site and may be utilized to alert patients and/or health care providers to the presence of pathogens at an early stage of infection, thereby providing for earlier intervention and improved recovery rates from infection.

Any source of HAI may be detected according to disclosed methods. For instance, while common bacterial sources such as Escherichia coli, Staphylococcus aureus, and Pseudomonas aeruginosa may be of particular interest in certain embodiments, disclosed methods are not limited to these bacteria. Other common sources of HAI that may be detected according to disclosed methods include, without limitation, other bacterial sources such as coagulase-negative staphylococci, Enterococcus spp., Enterobacter spp., Klebsiella pneumoniae, Proteus mirablis, Streptococcus spp., and so forth, as well as yeast, fungal, viral, and parasitic sources, as previously mentioned. In particular, it should be understood that the microorganisms that may be detected in accordance with the present disclosure are not particularly limited, and may include bacteria, yeast, fungi, mold, protozoa, viruses, etc. Several relevant bacterial groups that may be detected in the present invention include, for instance, gram negative rods (e.g., Entereobacteria); gram negative curved rods (e.g., vibious, Heliobacter, Campylobacter, etc.); gram negative cocci (e.g., Neisseria); gram positive rods (e.g., Bacillus, Clostridium, etc.); gram positive cocci (e.g., Staphylococcus, Streptococcus, etc.); obligate intracellular parasites (e.g., Ricckettsia and Chlamydia); acid fast rods (e.g., Myobacterium, Nocardia, etc.); spirochetes (e.g., Treponema, Borellia, etc.); and mycoplasmas (i.e., tiny bacteria that lack a cell wall).

Detection methods as disclosed herein utilize a sensor incorporating one or more biocompatible ISFETs in detection of pathogens that may cause HAI. More specifically, in the presence of a pathogen, the ion concentration in the in vivo vicinity of an ISFET may change, leading to a detectable change in conductivity or voltage between the source and drain of the ISFET. Detection of the change in electrical characteristics of the ISFET may alert a patient and/or medical personnel to existence of a pathogen at the site. Accordingly, detection of the change in ion concentration at the site may be utilized by medical personnel as a signal to initiate infection treatment. In addition, the particular species of pathogen and/or concentration may be determined upon further investigation of the site.

According to one embodiment, an ISFET may be utilized to directly measure changes in local pH due to the presence of pathogens, e.g., bacteria, in the local area of the ISFET sensor. For instance, an ISFET may be used to register abnormally low pH, i.e., acidic conditions, at the site of inquiry, which may be a sign of infection. The presence of certain pathogenic bacteria has been associated with acidic conditions. For example, the presence of P. aeruginosa may lead to a decrease in pH in the local in vivo environment (see, e.g., Saymen, et al., Applied Microbiology, 23:3, 509, 1972, and Shorrock, Worcester Polytechnic Institute, 2000). Certain Candida species (e.g., C. albicans) may also produce metabolites that decrease the pH of the local environment.

The presence of pathogens at a site may alternatively be indicated by an increase in local pH. For instance, bacteria of the genus Streptococcus may produce amines during metabolism, leading to an increase in local pH when the pathogen is present (see, e.g., U.S. Pat. No. 6,117,090 to Caillouette, which is incorporated herein by reference).

Corynebacterium urealyticum is a gram-positive commensal microorganism of the skin that is a known cause of nosocomial urinary infections. This bacterium produces urease that transforms urea into ammonia. Thus, the presence of this pathogen may be indicated by an increase in local pH, and a detected increase in pH may be indicative of, in such an embodiment, a nosocomial urinary tract infection.

The presence of certain Escherichia strains such as E. coli STa toxin have also been shown to cause a local alkalinization of tissue (see, e.g., McEwan, et al., Proceedings of the Royal Society of London. Series B, Biological Sciences, 234:1275 (1988), 219). Thus, a local increase in pH may be indicative of the presence of a pathogenic E. coli.

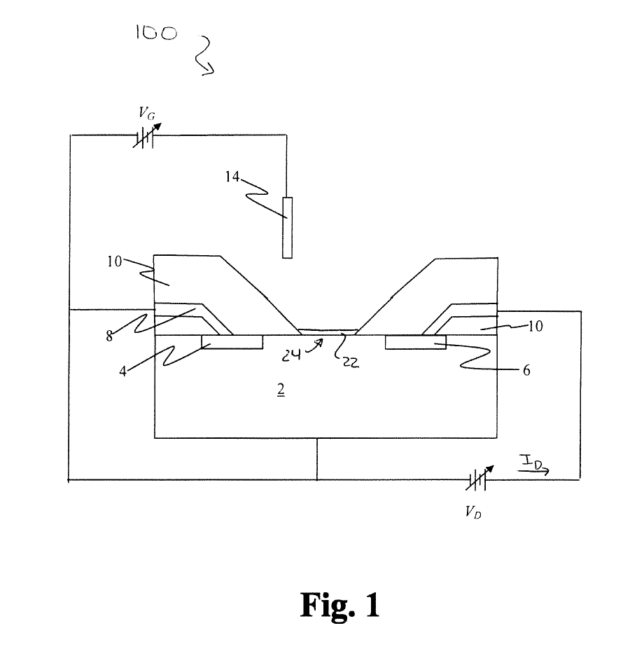

FIG. 1 illustrates a biocompatible ISFET 100 as may be utilized in one embodiment. ISFETs as disclosed herein may be biocompatible and as such may be safe for in situ sensing applications. In particular, at least those surface materials of an ISFET that may come into direct contact with tissue, fluids, and so forth following implantation may be formed of materials found acceptable for use in biomedical applications. More specifically, tissue-contacting components of an ISFET may be formed of materials that have been approved for utilization in applications associated with biological use or that possess no known health concern for in vivo use.

As may be seen, ISFET 100 includes an FET base 2. As utilized herein, the term ‘FET base’ generally refers to a semi-conductive support material within or upon which additional FET components may be affixed or formed. FET base 2 may be formed of a semi-conducting material, for example a p-type silicon. Mobile carriers (electrons or holes) inside of FET base 2 may be generated by impurity doping via, e.g., diffusion, implantation, and so forth. ISFET 100 may also include a drain 4 and a source 6. For example, when the base is formed of a p-type material, the drain 4 and source 6 will generally be n-type materials. In one embodiment, a semi-conducting material as may be incorporated in ISFET 100 may include an organic-containing semiconducting material. For instance, an organic-containing semiconducting material may be utilized in an undoped state and may be a p-type semiconductor or an n-type semiconductor. For example, an organic-containing semiconducting material may be an organic polymer e.g., a conjugated polymer. Conjugated polymers as may be utilized as disclosed herein may include, without limitation, polythiophene (PT), poly(p-phenylene) (PPP), poly(p-phenylene vinylene) (PPV), poly(2,5-thiophene vinylene) (PTV), polypyrrole (PPy) or C₆₀-Buckminster fullerene. An organic-containing semiconducting material may also include a conducting oligomer such as, for example, α-hexylthiophene (α-6T), pentacene and oligo-phenylene vinylene.

ISFET 100 also includes metal contacts 8 that contact the drain 4 and source 6, respectively, all of which are encapsulated in an insulative material 10, such as SiO₂, for example. In addition, ISFET 100 includes an electrode 14 that may contact the liquid being examined. The liquid will also make contact with the gate area 24.

In order to improve the sensitivity of the device, an ion sensitive layer 22 may be introduced to the device. The ion sensitive layer 22 may include, for example, an inorganic oxide, an inorganic nitride, or an inorganic oxynitride. In one embodiment, ion sensitive layer 22 may include silicon nitride, Si₃N₄ that may provide a charge blocking interface and improve pH response of the device. In other embodiments, ion sensitive layer 22 may include an amorphous metallic material including, without limitation, TiO₂, BaTiO₃, BaₓSr₁₋ₓ, TiO₃, Pb(ZrₓT₁₋ₓ)O₃, Ta₂O₅, SrTiO₃, BaZrO₃, PbTiO₃, LiTaO₃, and so forth. According to this embodiment, the ion-sensitivity of the device may be determined by the ionization and complexation of the surface hydroxy groups at the gate area 24.

Ion sensitive layer 22 may include materials to provide sensitivity for other specific ions, in addition to or alternative to protons and/or hydroxyls. For instance, in one embodiment, a poly(vinyl chloride) membrane including valinomycin may be utilized as an ion sensitive layer 22 to improve detection of potassium ions, the concentration of which may be increased in a local environment due to the presence of a pathogen.

Upon location in a field of inquiry, e.g., a surgical site, a wound site, a catheterization site, or so forth, the electrical characteristics of the ISFET may be monitored. Upon determination of a change in current or voltage due to a change in ion concentration, e.g., pH that may be caused due to the presence of a pathogen, intervention and/or additional examination of the site may be instituted.

The I_{D}/V_{G} characteristics of an ISFET device are generally that of the substructure on which it is based. For instance, characteristics may depend upon device design such as channel geometry and structure as well as on specific materials utilized and processing conditions, as is generally known in the art. Any suitable method may be utilized to determine the change in system characteristics upon a local change in ion concentration. For instance, when the applied gate bias potential is fixed, changes at the solution/ion sensitive layer 22 interface will be reflected in changes of the drain current (I_{D}). Optionally, the drain current may be maintained at a constant value through utilization of an operational amplifier that may directly control the applied gate bias potential with a negative feedback loop, according to known practices. In this case, the voltage output may vary with change in ion concentration in the local area.

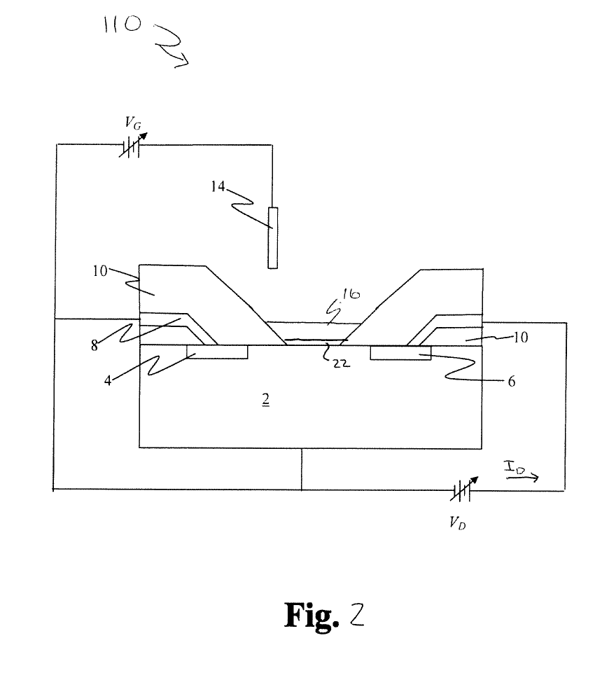

According to another embodiment, schematically illustrated in FIG. 2, a modified ISFET 110 may be utilized to determine the presence of a pathogen in the local area. More specifically, ISFET 110 may include a layer 16 in contact with the ion sensitive layer 22 within which may be entrapped one or more compounds that may improve detection of a change in local ionic concentration. For instance, in one embodiment, an enzyme specific for a metabolite or other compound that is produced by a pathogen may be incorporated in layer 16.

There are several ways to achieve the encapsulation of one or more compounds within a layer 16. For instance, in one embodiment layer 16 may be formed as a functionalized membrane, and a compound may be incorporated within the porous membrane. By way of example, one or more compounds may be entrapped within the matrix of a semi-permeable polymeric membrane used in formation of layer 16. A compound entrapped within layer 16 may recognize and interact with an expression product of a pathogen, e.g., a metabolite, or may interact with the pathogen itself. For example, upon interaction of a compound held in the layer 16 with a metabolite of a pathogen, an ionic product may be formed that may trigger a certain response in the ISFET, such as a voltage change, that may then be detected. This change in the output of the FET may be monitored in order to alert medical personnel to the presence of the pathogen in the local area.

Enzymes as may be included in layer 16 may act upon various types of substrates as may be expressed by HAI pathogens. As used herein, the term “substrate” generally refers to a substance that is chemically acted upon by an enzyme to form a product. In one embodiment, enzymes as may be incorporated in layer 16 may include hydrolases, lyases, transferases, and so forth. In some embodiments, the enzyme is a “hydrolase” or “hydrolytic enzyme”, which refers to enzymes that catalyze hydrolytic reactions. Examples of such hydrolytic enzymes include, but are not limited to, proteases, peptidases, lipases, nucleases, homo- or hetero-oligosaccharidases, homo- or hetero-polysaccharidases, phosphatases, sulfatases, neuraminidases and esterases. In one embodiment, for example, peptidases may be incorporated in layer 16. “Peptidases” are hydrolytic enzymes that cleave peptide bonds and often act upon shorter peptides. Examples of peptidases include, but are not limited to, metallopeptidases; dipeptidylpeptidase I, II, or IV; and so forth. In another embodiment, proteases may be incorporated in layer 16. “Proteases” are hydrolytic enzymes that cleave peptide bonds found in longer peptides and proteins. Examples of proteases that may be incorporated in later 16 according to the present disclosure include, but are not limited to, serine proteases (e.g., chymotrypsin, trypsin, elastase, PSA, etc.), aspartic proteases (e.g., pepsin), thiol proteases (e.g., prohormone thiol proteases), metalloproteases, acid proteases, and alkaline proteases. An enzyme as may be included in any of the disclosed devices may occur naturally or be synthetic.

Expressed substrates for hydrolytic enzymes contained in layer 16 include, for instance, acids, esters, amides, peptides, ethers, or other chemical compounds having an enzymatically-hydrolyzable bond. The enzyme-catalyzed hydrolysis reaction may, for example, result in a hydroxyl or amine compound as one product, and a free phosphate, acetate, etc., as a second product. Specific types of substrates may include, for instance, proteins or glycoproteins, peptides, nucleic acids (e.g., DNA and RNA), carbohydrates, lipids, esters, derivatives thereof, and so forth. For instance, some suitable substrates for peptidases and/or proteases may include peptides, proteins, and/or glycoproteins, such as casein (e.g., β-casein, azocasein, etc.), albumin (e.g., bovine serum albumin (BSA)), hemoglobin, myoglobin, keratin, gelatin, insulin, proteoglycan, fibronectin, laminin, collagen, elastin, and so forth.

For instance, in one particular embodiment, an enzyme may be incorporated in a layer 16 that may interact with lactic acid produced during metabolism of bacteria. Lactic acid is produced as a major component of metabolism in a large number of pathogenic bacteria. The genera that comprise these lactic acid bacteria (LAB) include Lactobacillus, Leuconostoc, Pediococcus, Lactococcus, and Streptococcus, Aerococcus, Carnobacterium, Enterococcus, Oenococcus, Teragenococcus, Vagococcus, and Weisella. Accordingly, inclusion of a compound in a layer 16 that may improve detection of lactic acid may be utilized to provide detection of a large number of potential pathogens. For instance, an enzyme for lactic acid such as lactate oxidase may be included in layer 16. Lactate oxidase oxidizes lactic acid to produce hydrogen peroxide, the production of which will cause a decrease in local pH and a corresponding alteration in an output signal from an ISFET at the local environment.

Layer 16 may be formed according to any suitable process so as to form a semi-permeable layer that may entrap enzymes, cofactors, or other compounds in the layer while allowing access to the compounds by the materials in the surrounding solution. For instance, in one embodiment, layer 16 may be a multilayer membrane that may encapsulate enzymes between layers. For instance, cellulose acetate and copolymers of cellulose acetate may be utilized in formation of a multilayer membrane. Perfluorosulfonic acid polymers, such as Nafion™ have also been utilized to form multi-layer membranes for enzyme modified ISFETs.

Layer 16 may also be in the form of a polymeric matrix. For instance, a semi-porous hydrogel matrix, e.g., a polyvinyl-based or polyacrylamide-based hydrogel, may be utilized to encapsulate one or more enzymes at the ISFET gate. In the presence of a pathogen, a substrate specific for the enzyme may be expressed by the pathogen and, as the polymeric matrix is semi-permeable, the substrate may diffuse through the matrix to contact the encapsulated enzyme and form a product, the presence of which may alter the current or voltage of the ISFET.

For instance, a polyvinyl-based hydrogel matrix including polyvinyl alcohol and polyvinyl pyrrolidine may be utilized as is described in U.S. Pat. No. 5,134,057 to Kuypers, et al., which is incorporated herein by reference. By way of example, layer 16 may be applied to the desired surface through application of a solution including the polymers, the compound(s) to be held in the matrix, crosslinking agents, such as a photoactive crosslinking agent or glutaraldehyde, and any buffers. The solution may be applied to the ion sensitive surface 22 and crosslinked, for instance through application of UV light.

In another embodiment, a polyacrylamide-based hydrogel matrix may be utilized to entrap a compound that may improve detection of local changes in ion concentration. For instance, a polyacrylamide-based hydrogel matrix as is disclosed in U.S. Pat. No. 4,812,220 to Iida, et al., which is incorporated herein by reference, may be utilized. According to this particular embodiment, a solution containing acrylamide monomers, a crosslinking agent (e.g., N,N′-methylenebisacrylamide), an initiator (e.g., riboflavin and peroxodisulfate), and a polymerization promoter (e.g., N,N,N′,N′-tetramethylethylenediamine) may be added to solution including one or more enzymes and polymerized with light under a nitrogen blanket.

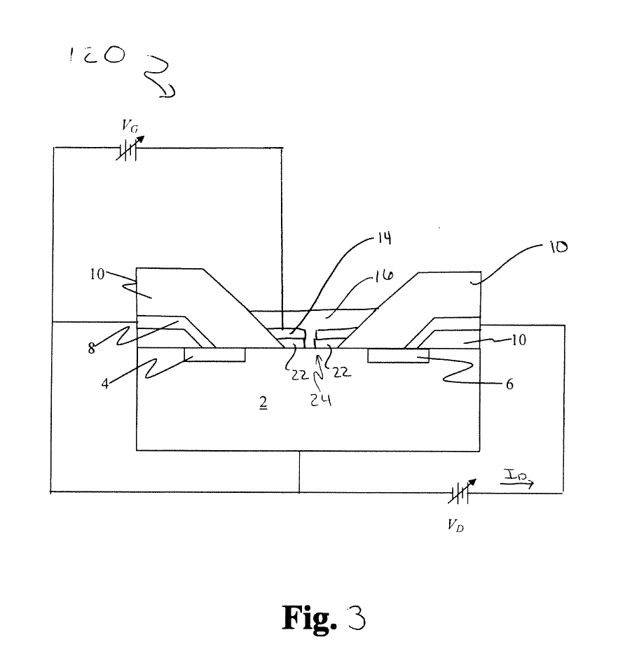

According to disclosed methods, the products of a reaction, e.g., an enzyme catalyzed reaction, will change the concentration of protons (H⁺) or hydroxyls (OH), and therefore the pH within the layer 16. As the buffer capacity of the solution at the gate depends upon the pH of the solution, the response of the ISFET may be nonlinear and the dynamic range of the sensor may depend upon the composition of the solution. Accordingly, in one embodiment, illustrated in FIG. 3, a static enzyme sensor may be utilized to measure the pH inside the layer 16 and maintain it at a constant level. Specifically, a static enzyme sensor measures the pH inside the membrane and controls it via generation of H⁺ or OH⁻, as required, at the electrode 14, e.g., a gold or platinum electrode, which is near the gate area 24, as shown. In this embodiment, the ionic reaction products of a reaction are continually neutralized, and the generating current required to maintain the pH at the set point becomes the output signal of the ISFET. This current may be linearly related to the substrate concentration and thus the pathogen concentration in the local area. In addition, the output of the ISFET in this embodiment will be independent of the buffer capacity of the sample.

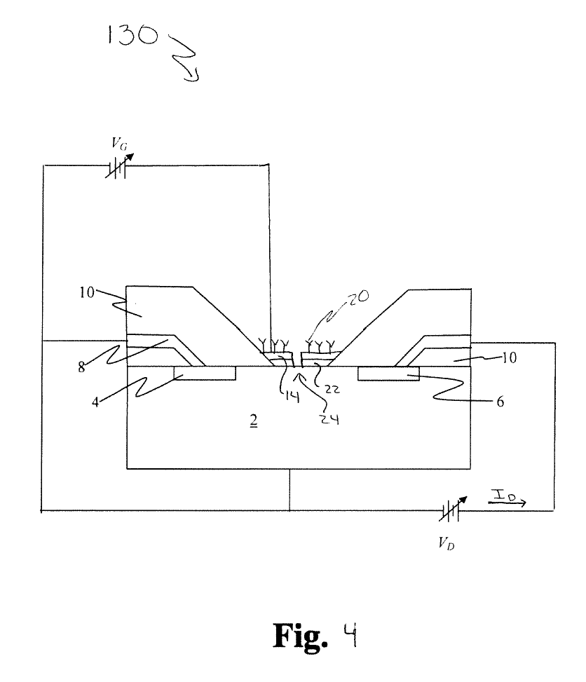

According to another embodiment, illustrated in FIG. 4, a probe 20 for a pathogen may be located at the gate area 24 of an ISFET to improve detection of the change in local ion concentration due to the presence of the pathogen. A probe as utilized herein generally refers to a molecule able to react with another molecule to form a complex and/or induce a secondary reaction. By way of example, a probe 20 as may be bound incorporated in an ISFET may include an enzyme, antibody, antigen, carbohydrate, peptide, DNA fragment, RNA fragment or oligonucleotide. Examples of probes as may be utilized are described in U.S. Pat. Nos. 5,063,081 to Cozzette, et al., 5,627,079 to Gardella, Jr., et al., 6,060,327 to Keen, the contents of which are incorporated herein by reference. Probes may be purified from a living source or may be made by any method of synthesis known in the art.

In one embodiment, a product of a pathogen to be detected may bind or react with the probe. A product may therefore include enzymes, antibodies, antigens, peptides, DNA fragments, RNA fragments, oligonucleotides and so forth.

In one embodiment, a probe 20 may be a specific binding member for an expression product of a pathogen or an extracellular component of a pathogen. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies, and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

In another embodiment, a probe 20 may be a compound that may be metabolized by a pathogen to produce detectable ions. For instance, certain carbohydrates such as mannitol, glucose, lactose, and so forth, may be metabolized by certain pathogens to form ionic products. For example, fermentation of mannitol by S. aureus and some strains of S. saprophyticus are known to cause a detectable pH change in the local environment. Lactose fermentation by coliform bacteria and E. coli may also produce a detectable change in local pH. According to this particular, embodiment, a carbohydrate may be bound as a probe 20. The probe 20 may be ingested by the bacteria to produce detectable ions by metabolism of the carbohydrate probe 20.

In another embodiment, a probe 20 may be utilized to bind the pathogen itself at the ISFET, and thus hold and concentrate the source of the ion production near the gate of the ISFET. For instance, a probe 20 may be a specific binding member for a surface protein or carbohydrate of the pathogen.

In yet another embodiment, a combination of probe types may be utilized. For instance, a first probe may bind a pathogen to a surface of the ISFET, and a second probe may include a carbohydrate to provide a fermentation source for the production of detectable ions in the local environment.

A probe 20 may generally be bound to a surface of an ISFET, for instance a surface of electrode 14 as shown in FIG. 4 using any of a variety of well-known techniques. For instance, covalent attachment of the specific binding members to the surface of electrode 14 may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer as the surface of the ISFET may contain a relatively high surface concentration of polar groups. In addition, the surface of the ISFET may be capable of direct covalent linking with a probe, e.g., a protein, without the need for further modification. For example, specific covalent linkage between cysteine residues of a probe 20 may directly bond an electrode 14 of gold.

In another embodiment, the first step of conjugation of a probe to a surface of an ISFET is activation of carboxylic groups on the surface using carbodiimide. In the second step, the activated carboxylic acid groups are reacted with an amino group of a probe, e.g., an antibody or an enzyme, to form an amide bond. The activation and/or probe coupling may occur in a buffer, such as phosphate-buffered saline (PBS) (e.g., pH of 7.2) or 2-(N-morpholino) ethane sulfonic acid (MES) (e.g., pH of 5.3). The resulting probes may then be blocked with ethanolamine, for instance, to form the probe conjugate.

Besides covalent bonding, other attachment techniques, such as physical adsorption, may also be utilized. For example, a probe, such as an enzyme probe, may be immobilized on a surface of the ISFET via ionic interaction or physical force.

In another embodiment, a probe may be crosslinked to the surface of an ISFET. For instance, a probe and amino groups of a stabilizing protein such as albumin may be crosslinked, for instance with a glutaraldehyde crosslinking agent or a photoactive crosslinking agent, to bind the probe to the surface of an ISFET.

Upon exposure of probes 20 to a specific reactant for the probes, e.g., a specific binding member, interaction between the two may cause a change in impedance of the electrode 14. The alteration in impedance, e.g., an increase in current through the device, is indicative of the analyte, which may be, for example, the pathogen itself, or an expression product of the pathogen.

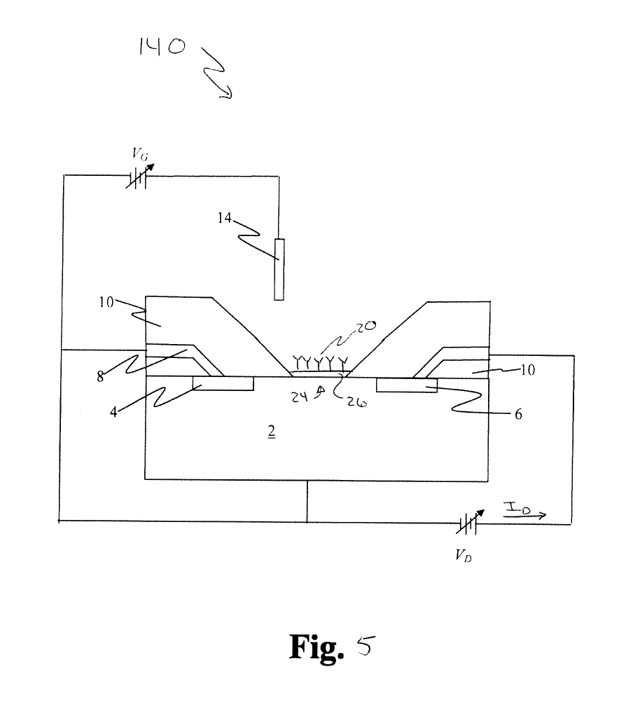

In yet another embodiment, illustrated in FIG. 5, a probe 20, e.g., a specific binding member, may be bound on an insulative layer 26 that may be applied over a portion of the semi-conductive FET base 2.

According to this particular embodiment, binding of an analyte to a probe 20 may change the device's surface potential by any combination of the following contributions:
1) Ionic changes of the probe upon formation of a probe/analyte conjugate
2) Formation of a binding-induced dipole moment
3) Modification of energy distribution and/or
4) Modification of density surface states.

Any change of surface potential in turn leads to a change in the band structure and charge distribution in the semiconductor material of FET base 2 directly changing the lateral conductivity due to the close proximity to the surface. This effect is highly sensitive to even minor changes in surface potential. According to one embodiment, a probe 20 may be immobilized on top of the insulative layer 26 according to any suitable binding methodology. For instance, probes 20 may be bound to layer 26 using carboxylic, amino, aldehyde, bromoacetyl, iodoacetyl, thiol, epoxy and other reactive or linking functional groups, as well as residual free radicals and radical cations, through which a protein coupling reaction may be accomplished, and as discussed previously. According to another embodiment, covalent binding of a probe 20 may be achieved through a siloxane linkage, for instance in those embodiments in which insulative layer 26 is a silicon dioxide layer. Another approach for the functionalization is the physisorption of a polymer layer, such as functionalized molecules.

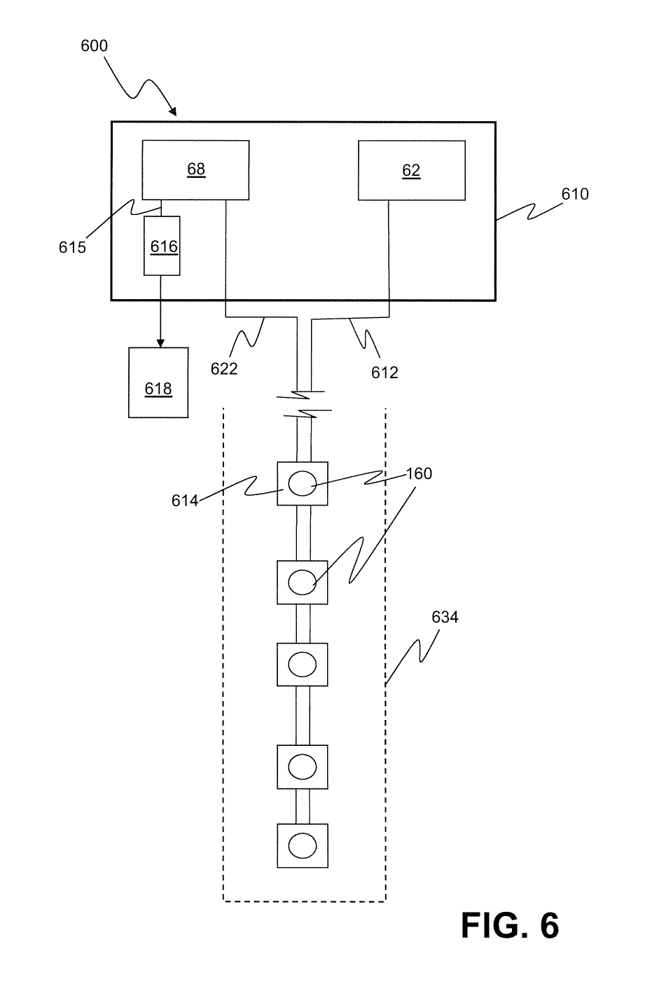

In accordance with the present technology, one or more ISFETs may be utilized as a portion of a sensor that may monitor a potential HAI infection site for a change in ionic concentration that may be indicative of the presence of one or more disease causing pathogens. While not required, in one preferred embodiment, a sensor may be portable. For example, a sensor as disclosed herein may be a portable device, one embodiment of which is schematically illustrated in FIG. 6. As may be seen in FIG. 6, sensor 600 may include several components that may be housed within an enclosure 610.

Enclosure 610 may be, for example, a molded plastic enclosure of a size so as to be easily carried by or attached to a patient. For instance, enclosure 610 may include clips, loops, or so forth so as to be attachable to a patient's clothing or body. In one embodiment, enclosure 610 may include a biocompatible adhesive at a surface, and may be adhered directly to a patient's skin. In general, enclosure 610 may be relatively small, for instance less than about 10 cm by about 8 cm by about 5 cm, so as to be inconspicuously carried by a patient and so as to avoid impedance of a patient's motion. Enclosure 610 may completely enclose the components contained therein, or may partially enclose the components contained therein. For example, enclosure 610 may include an access port (not shown) that may provide access to the interior of enclosure 610. In one embodiment, an access port may be covered with a removable cover, as is known in the art.

A first component as may be held within enclosure 610 is power supply 62 that may be configured in one embodiment to supply power to ISFET 160 as well as other of the operational components as will be later described. In an exemplary configuration, power supply 62 may correspond to a battery, however those of ordinary skill in the art will appreciate that other power supplies may be used including those that may be coupled to an external alternating current (AC) supply so that the enclosed power supply may include those components necessary to convert such external supply to a suitable source for the remaining components requiring a power source.

As previously noted, power supply 62 may be configured in one embodiment to supply power to ISFET 160 via line 612. Line 612 is configured to extend externally from enclosure 610 to an ISFET 160 that may be located in the field of inquiry, e.g., within a surgical site or other wound. One or more ISFETs may be located at a site according to any suitable method. For instance, one or more ISFETs may be simply located at the site of interest during a medical procedure. For instance, prior to closing a surgical site or at the time of placement of a catheter or an endotracheal tube, a portion of sensor 600 including an ISFET 160 may be located at the site.

In general, ISFET 160 may be mounted on a biocompatible support 614. Materials as may be utilized in forming biocompatible support 614 may include, without limitation, low viscosity thermoplastic polymers such as polymethyl methacrylate (PMMA), polyoxymethylene (POM), polyamide (PA), or polycarbonate, as well as reaction resins based on methacrylates, silicones and caprolactames. However, many more biocompatible materials as are generally known in the art may be utilized to form support 614.

ISFET 160 may be of any convenient size and shape for location at a site of inquiry. For instance, support 614, carrying an ISFET 160 may be less than about 1 mm in height and have a width of less than about 100 μm, in one embodiment.

A sensor as disclosed herein may include a plurality of ISFETs 160. For instance, in the illustrated embodiment, sensor 600 may include a plurality of ISFETs 160 in series. This is not a requirement, however, and in other embodiments, a plurality of ISFETs may be provided in any suitable combination.

A plurality of ISFETs 160 of a sensor may be the same or different. For instance, each ISFET may be associated with the same or different probes, enzymes, or other compounds that may improve detection of a local change in ion concentration. Accordingly, a single sensor may produce a detectable change in output signal due to the presence of a plurality of different possible pathogens at a detection site. For instance, by combining many such ISFET units to whole arrays and functionalizing them with individual probes, for instance by using lithographic techniques, whole ensembles of pathogens and/or pathogen expression products may trigger a detectable change in local ion concentration levels.

In various embodiments, a barrier 634 may be included with a system to protect ISFETs 160 from an external environment. For instance, barrier 634 may be a semi-permeable barrier defining a porosity that may allow an expression product from a pathogen to pass through barrier 634 and interact with ISFET 160, while preventing passage of other materials. For instance, barrier 634 may prevent a pathogen from contacting ISFET 160. Barrier 634 may keep other potential contaminants away from ISFET 160 as well. For instance, barrier 634 may prevent materials that may be common at the detection site, e.g., toxins, ECM components, leukocytes, red blood cells, and so forth, from contacting and/or blocking communication between the targeted expression product of a pathogen and ISFET 160.

Barrier 634 may be, for instance, a semi-permeable porous membrane having a porosity to allow materials less than about 0.2 μm across the membrane, with a preferred pore size generally depending upon the size of expression products that are targeted by the sensor. Semi-permeable membrane 634 may be, for example, derived from a water insoluble, water wettable cellulose derivative, such as cellophane, cellulose acetate, cellulose propionate, carboxyethyl cellulose, and so forth; insolubilized gelatin; partially hydrolized polyvinyl acetate; or polyionic film forming compositions such as polysulfonated anionic polymers or ionically linked polycationic polymers, such as marketed by Amicon Company. Barrier 634 may surround a series of ISFETs 160, as shown, and may be attached to line 612 at a distance from the terminus of or the sensor 600 or optionally may be attached to another component of a sensing system.

Housed within enclosure 610 is a detector 68 coupled to line 622. Detector 68 may include a microprocessor or other signal processor configured to evaluate the strength or other characteristics of the output signal received over 622 to, e.g., correlate the signal to a predetermined trigger level beyond which the potential of an infectious concentration of pathogens at the detection site, e.g., greater than about 10⁻⁵ CFU/mL, is likely. Upon detection of a change in output that exceeds this predetermined set point, a detection signal may be produced that may be coupled to line 615 for passage to a signaling device 616. Accordingly, if the detection signal reaches a predetermined threshold value corresponding to a high possibility existence of a local infection, for instance corresponding to a change in local pH of at least about 0.3 pH units, or greater in other embodiments, for example a change in local pH of at least about 0.4 pH units, or at least about 0.5 pH units, a detectable signal may be initiated at signaling device 616. In an exemplary configuration, a detectable signal may initiate a visible or audible signal within or at the surface of the enclosure 610 by way of signaling device 616 that may be detected by the wearer. For instance, a visible signal may optionally include utilization of a liquid crystal diode (LCD) device, or an equivalent thereof, that may provide the signal as a readable output. For example, a visual signal may be provided at a surface of the enclosure 610 as an instruction such as, for instance, “CALL YOUR DOCTOR”, “VISIT HOSPITAL,” or so forth.

In addition to or alternative to a visual and/or audible signal at the enclosure 610 itself, signaling device 616 may include a transmitter portion that, upon initiation of the detectable signal, may transmit an electromagnetic signal to receiver 618. Receiver 618 may be remote from the signaling device 616. For instance, receiver 618 may be on the wearer's body at a distance from the signaling device 616, at a location apart from the wearer's body that may be conveniently chosen by the wearer, e.g., within the wearer's home, office, or so forth, or may be at a monitoring facility, for instance at a medical facility, such that appropriate medical personal may be quickly informed of the change in status of the patient's site of inquiry. In alternative embodiments, the detectable signal may be transmitted to multiple receivers, so as to inform both the wearer and others (e.g., medical personnel) of the change in status of a site. Transmission of a signal to a remote site may be carried out with a radio frequency transmission system or with any other wireless-type transmission system, as is generally known in the art. For instance, a wireless telephone or internet communications system may be utilized to transmit a signal to a remote location according to known methods.

Wireless transmission systems as may be utilized in conjunction with disclosed devices and methods may include, for example, components and systems as disclosed in U.S. Pat. Nos. 6,289,238 to Besson, et al., 6,441,747 to Khair, et al., 6,802,811 to Slepian, 6,659,947 to Carter, et al., and 7,294,105 to Islam, all of which are incorporated in their entirety by reference.

While the subject matter has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of the appended claims and any equivalents thereto.

1. A method for detecting a pathogen that is a source of a hospital acquired infection comprising:
locating an ion sensitive field effect transistor in an in vivo environment;
transmitting an output signal from the ion sensitive field effect transistor to a detector;
detecting a change in the output signal of the ion sensitive field effect transistor due to the presence of the pathogen in the environment.

2. The method according to claim 1, further comprising transmitting information regarding the change in the output signal from the ion sensitive field effect transistor in the in vivo environment to a receiver.

3. The method according to claim 2, wherein the information is transmitted to the receiver by use of a wireless transmission system.

4. The method according to claim 1, wherein the ion sensitive field effect transistor is located in a surgical site.

5. The method according to claim 1, wherein the ion sensitive field effect transistor is located in a wound.

6. The method according to claim 1, wherein the ion sensitive field effect transistor is located at a catheterization site.

7. The method according to claim 1, further comprising providing information regarding the change in the output signal of the ion sensitive field effect transistor as at least one of a visual and an audible signal.

8. The method according to claim 1, further comprising locating a second ion sensitive field effect transistor in the in vivo environment.

9. The method according to claim 8, wherein the first ion sensitive field effect transistor and the second ion sensitive field effect transistor are in series.

10. The method according to claim 1, further comprising contacting an expression product of the pathogen with a compound that is associated with the ion sensitive field effect transistor.

11. The method according to claim 1, further comprising binding the pathogen or an expression product of the pathogen with a probe that is associated with the ion sensitive field effect transistor.

12. A device for in vivo detection of a pathogen that is a source of a hospital acquired infection comprising:
a power source;
a biocompatible ion sensitive field effect transistor for inserting into an in vivo environment, the biocompatible ion sensitive field effect transistor being in electrical communication with the power source;
a detector in electrical communication with the ion sensitive field effect transistor for detecting a change in the output signal of the biocompatible ion sensitive field effect transistor due to the presence of the pathogen in the environment; and
a signaling device in electrical communication with the detector for emitting a signal upon detection of the change in the output signal of the biocompatible ion sensitive field effect transistor in an environment.

13. The device of claim 12, wherein the power source, the detector, and the signaling device are all contained within a portable enclosure, the portable enclosure further comprising a connecting device at an external surface of the enclosure.

14. The device of claim 13, wherein the connecting device is for attaching the enclosure to a piece of clothing.

15. The device of claim 13, wherein the connecting device is for attaching the enclosure to a wearer's skin.

16. The device of claim 12, the device further including a transmitter for transmitting a signal containing information regarding the change in the output signal of the biocompatible ion sensitive field effect transistor to a receiver.

17. The device of claim 16, wherein the transmitter is a wireless transmitter.

18. The device of claim 12, further comprising a compound associated with the ion sensitive field effect transistor, wherein an expression product of the pathogen interacts with the compound to form an ion.

19. The device of claim 18, wherein the compound is contained within a layer of the ion sensitive field effect transistor.

20. The device of claim 18, wherein the compound is bound to a surface of the ion sensitive field effect transistor.

21. The device of claim 18, wherein the compound is an enzyme.

22. The device of claim 18, wherein the expression product is a metabolite.

23. The device of claim 12, the device further comprising a probe for the pathogen or an expression product of the pathogen.

24. The device of claim 23, wherein the probe is a specific binding partner of the expression product of the pathogen.

25. The device of claim 12, further comprising a semi-permeable barrier surrounding the ion sensitive field effect transistor.

26. The device of claim 12, wherein the signaling device emits a plurality of signals upon detection of the pathogen in an environment.

27. The device of claim 12, the device further comprising a plurality of ion sensitive field effect transistors.
